(19) United States
(12) Patent Application Publication
(10) Pub. No.: US 2025/0014112 A1
(43) Pub. Date: Jan. 9, 2025

(54) METHOD AND SYSTEM FOR AUTOMATED DATA INGESTION IN A REAL-TIME

(71) Applicant: INNOVACCER INC., San Francisco, CA (US)

(72) Inventors: Tanay RAGHUVANSH, New Delhi (IN); Mridul SARAN, Bareilly (IN); Amit CHOWHAN, Ghaziabad (IN); Vaibhav SHARMA, Noida (IN)

(21) Appl. No.: 18/420,434

(22) Filed: Jan. 23, 2024

(30) Foreign Application Priority Data
Jul. 8, 2023 (IN) 202311046007

(51) Int. Cl. G06Q 40/08; G16H 10/60

(52) U.S. Cl. CPC G06Q 40/08; G16H 10/60

(57) ABSTRACT

Methods and systems for automatically and dynamically ingesting healthcare data from a plurality of data sources into a single unified ingestion database, in a real-time include a plurality of client computing devices for capturing healthcare data of a patient. A central server is connected to each client computing device. A memory of the server includes one or more ingestion data tables to store healthcare data received from the client computers and/or a plurality of data sources in a predetermined data-pattern. An ingestion module includes algorithms to process the input healthcare data. The central server receives the input healthcare data from the client computing devices and the ingestion module identifies an ingestion data-pattern for the ingestion data table and corresponding mapping codes, in accordance with the historical healthcare data received from the plurality of data-sources. The input healthcare data is processed and ingested within the ingestion table using the mapping codes.

### CROSS REFERENCE TO RELATED APPLICATION

This application claims benefit of India application No. 202311046007 filed Jul. 8, 2023, the entire disclosure of which is incorporated herein by reference.

### TECHNICAL FIELD

The present disclosure in general relates to data ingestion, and in particular relates to implementation of real time ingestion of data pertaining to insurance claims data from multiple data sources.

### BACKGROUND

Many business sectors, including the healthcare sector, deal with complex data and data management. Integrating information from multiple sources can be complex and time consuming, particularly, when the information and data are recorded on paper or electronic documents in different formats. For example, the healthcare sector deals with challenges of integrating big data files to process insurance claims. The data files of the healthcare insurance sector may include patient records, information about healthcare professionals, billing information, insurance plans, tenure, medical insurance claims, pharmacy insurance claims, etcetera. The processing of medical insurance claims may include huge documentations and a considerable number of correspondences between patients, healthcare providers and insurance companies over a substantial period of time, for example several weeks or months.

In the healthcare sector, the information pertaining to medical insurance claims relative to accumulated bills is typically named as ‘claims data’ and this information is recorded in a claims data file. A typical claims data file comprises a number of columns and rows, wherein each column and each row has recorded information in the form of values, texts, codes, etc. A claims data file has to be processed for billing and payment reimbursements purposes from insurance companies. Since the claims data contains a very large number of records, its processing and settlement becomes a cumbersome process and requires people with high skill and experience to both ingest as well as examine the resulting data for quality check. Any errors in data, such as incorrect information, incorrect amount values, patient IDs, doctor IDs, codes etc., creates further complications. Consequently, the medical insurance claims data analytics not only becomes a lengthy process but may also result in mismanagement or loss of revenue. Therefore, both the healthcare providers and insurance companies are required to carefully examine the claims data files to maintain a balance between each other while avoiding any data mismanagement.

Conventionally, the claims data is examined manually and are therefore prone to errors resulting in incorrect results while performing analytics on such data. A solution to eliminate the manual process of claims data inspection is to provide a computer-implemented mapping process wherein data is ingested and mapped to a given schema for inspection. However, such a solution requires the claims data to be supplied in a standard format and hence, lacks the capability of handling claims data having different data formats. In the current scenario, every insurance company supplies claims data in a format specific to that company alone and hence, this process has become much more complex. Further, the heterogeneous data sources can make the processing more complicated, with separate data processing for the data from each data source. Such a process of obtaining, importing, and processing data for later use or storage in a database, is known as data ingestion.

In view of the above, the present disclosure as disclosed herein, aims to provide a novel system and method for providing automated ingestion of claims data in real time.

### SUMMARY

In one aspect of the present disclosure, a system for automatically and dynamically ingesting healthcare data, particularly, insurance claims data from a plurality of data sources into a single unified ingestion database, in a real-time, is disclosed. The system comprises a plurality of client computing devices, each configured to be used to capture one or more healthcare data of a patient, or a plurality of patients in a predetermined format. The system further includes a central server adapted to be connected to each of the client computing devices by using one or more connection mediums. The central server is generally a yet another computing device having a first communication interface adapted to receive input healthcare data and/or requests from the one or more client computing devices, the healthcare data generally pertaining to one or more insurance claim data related to the patients from one or more sources. The central server further includes a first processor and a first memory configured to execute one or more first programming instructions embodied thereon. The first memory includes one or more ingestion data tables adapted to store healthcare data received from the one or more client computers and/or a plurality of data sources in a predetermined data-pattern. The central server further includes a data receiving component adapted to receive a plurality of historical data pertaining to the healthcare data, from a plurality of data sources and/or computing devices and/or other central servers.

The central server further includes an ingestion module comprising one or more algorithms adapted to be processed by the first processor, each adapted to process the input healthcare data so as to ingest them into the ingestion tables. The ingestion module comprises a machine learning based processing module adapted to upgrade the ingestion module dynamically. Particularly, the machine learning based processing module is configured to access and process the historical healthcare data received from the plurality of data-sources so as to train the ingestion module dynamically. In operation, the central server receives the one or more input healthcare data from the one or more client computing devices. Thereafter, the ingestion module is configured to identify an ingestion data-pattern for the ingestion data table and corresponding mapping codes, in accordance with the historical healthcare data received from the plurality of data-sources. Thereafter, the input healthcare data is processed and ingested within the ingestion table by using the mapping codes.

Potentially, the central server furthermore includes an analytics module adapted to report one or more analysis from the healthcare data via a reporting module.

Further potentially, the central server includes a reporting unit adapted to display an output of the analysis using the analytics module.

Particularly, the reporting unit includes one or more display units selected from but not limited to an interactive touch display unit, a LED Monitor, CRT Monitor, and any other suitable display unit known in the art.

Generally, the healthcare information includes one or more information related to the insurance data of the patient, selected from one or more of but not limited to insurance details, diagnostic guidelines, diagnostic history of the patient, claims history, amounts, and the like.

Potentially, the connection interface is a wired communication interface selected from one or more of but not limited to USB, HDMI, CSI, LAN, and the like.

Alternatively, the connection interface is a wireless communication interface selected from one or more of but not limited to wi-fi, Bluetooth, hotspot, internet, intranet, wlan, and the like.

In another aspect of the present disclosure, a method for automatically and dynamically ingesting healthcare data received from a plurality of client computing devices in a unified form, in a real-time, is disclosed. The method includes receiving one or more input healthcare data, from one or more client computing devices, at a central server. The method further includes receiving plurality of historical healthcare information data, at the central server. Thereafter, the method includes processing the received input healthcare data, by first selecting an ingestion module, in accordance with the plurality of historical healthcare data and implementing programming instructions embodied thereon to determine an ingestion data-pattern. The method furthermore includes generating one or more mapping codes on the basis identified ingestion data-patterns followed by ingesting the input healthcare data in accordance with the identified data pattern by using the generated one or more mapping codes.

Potentially, the generation of mapping codes includes generating a table mapping code using a table mapper sub-module, column mapping code by using a column mapper sub-module, and a row mapping code using a row mapper sub-module.

Particularly, the method further includes updating the ingestion data-pattern in real time.

Further, the method includes validating the mapping of the ingested healthcare data within the ingested data table by using a validation module.

Potentially, the method of generating ingestion data patterns includes providing an initial training data within the ingestion table; receiving at the central server, at least one of historical claim data from one or more of a plurality of data sources, connected thereto; analyzing the received historical claim data to configure the table mapper and/or column mapper and/or row mapper to predefine one or more rules and/or schema for the tables and/or columns and/or rows respectively; and combining the identified rules and/or schemas to determine the data ingestion data pattern.

Further potentially, the method includes upgrading the ingestion module, in accordance with a machine learning sub-module at the back-end server.

Numerous additional features, embodiments, and benefits of the methods and apparatus of the present disclosure are discussed below in the detailed description which follows.

### BRIEF DESCRIPTION OF DRAWINGS

The accompanying drawings illustrate various embodiments of systems, methods, and other aspects of the disclosure. Any person having ordinary skill in the art will appreciate that the illustrated element boundaries (e.g., boxes, groups of boxes, or other shapes) in the figures represent one example of the boundaries. It may be that in some examples, one element may be designed as multiple elements or that multiple elements may be designed as one element. In some examples, an element shown as an internal component of one element may be implemented as an external component in another, and vice versa. Furthermore, elements may not be drawn to scale.

FIG. 1 illustrates a system block diagram of an automatic data ingestion system according to the present disclosure.

FIG. 2 depicts a block diagram illustrating an exemplary functionality of a mapper sub-module, in accordance with the present disclosure.

FIG. 3 shows a flow chart illustrating a method of automatically ingesting healthcare data in real time, according to the present disclosure.

FIG. 4 depicts an exemplary flowchart illustrating a method of identifying ingestion data-pattern, according to the present disclosure.

Various embodiments will hereinafter be described in accordance with the appended drawings, which are provided to illustrate, and not to limit the scope in any manner, wherein like designations denote similar elements, and in which:

### DETAILED DESCRIPTION

The present subject matter is best understood with reference to the detailed figures and description set forth herein. Various embodiments are discussed below with reference to the figures. However, those skilled in the art will readily appreciate that the detailed descriptions given herein with respect to the figures are simply for explanatory purposes as the methods and systems may extend beyond the described embodiments. For example, the teachings presented, and the needs of a particular application may yield multiple alternate and suitable approaches to implement the functionality of any detail described herein. Therefore, any approach may extend beyond the particular implementation choices in the following embodiments described and shown.

The present application provides an automatic healthcare data ingestion system for dynamically ingesting healthcare data within a central unified ingestion database. The system further enables visualizing various analytics related to the ingested data onto a reporting unit, preferably in real time. The system is further adapted to auto upgrade the data pattern of the unified database in real time. The system is generally provided with a plurality of historical data in combination with an ingestion module provided at the central server. In preferred embodiment, the system may be in the form of a web-based automated service accessible on a generally known client computing device.

Particularly, the system of the present subject matter is adapted to the healthcare data within a unified ingestion database accurately and automatically ingest while considering all the possible formatting/schema factors in combination with historical data of the patients, which may be utilized for the purpose of determining the data-pattern of the unified ingestion data tables. Additionally, the system of the current disclosure enables machine learning and artificial intelligence-based probe orientation guidance to enable an accurate identification of data-patterns/formats/schemas by using one or more predetermined analysis and/or classification algorithms. It is to be understood that unless otherwise indicated, this disclosure need not be limited to applications for healthcare data. As one of ordinary skill in the art would appreciate, variations of the disclosure may be applied to other databases from any domain. Moreover, it should be understood that embodiments of the present disclosure may be applied in combination with various other management systems such as hospital management, patient management, facility management systems, access management systems, human resource management system, occupational management systems, clinical systems, and the like, for various other possible applications. It must also be noted that, as used in this specification and the appended claims, the singular forms “a,” “an” and “the” include plural referents unless the context clearly dictates otherwise. Thus, for example, the term “a data-set” is intended to mean a single dataset or a combination of datasets, “an algorithm” is intended to mean one or more algorithms for the same purpose, or a combination of algorithms for performing different program executions.

References to “one embodiment,” “an embodiment,” “at least one embodiment,” “one example,” “an example,” “for example,” and so on, indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in an embodiment” does not necessarily refer to the same embodiment.

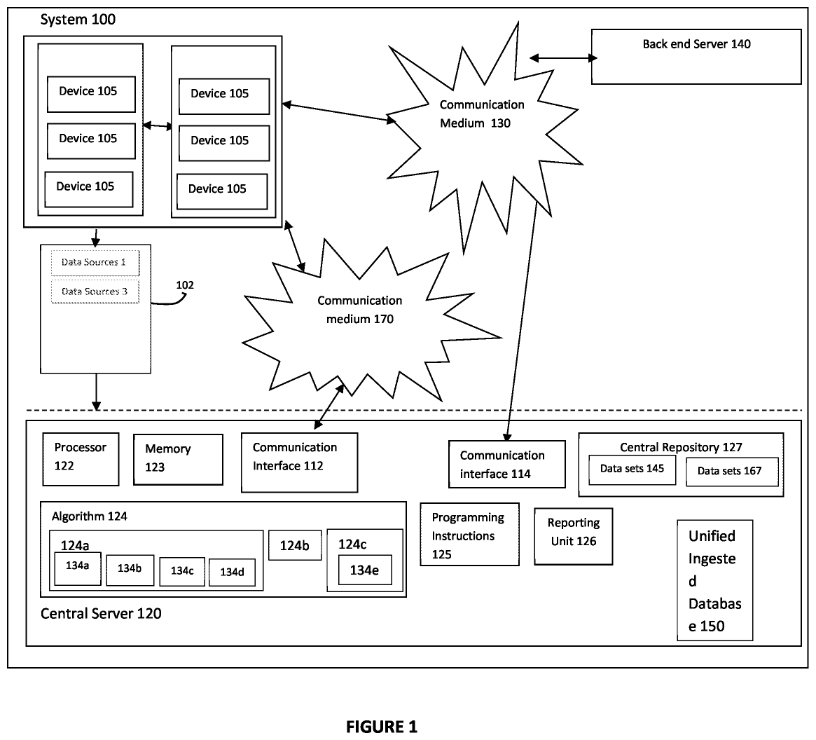

FIG. 1 is a system block diagram of an automatic healthcare data ingestion system 100 according to the present disclosure adapted to dynamically ingest healthcare data from a plurality of formats within a unified ingested database 150, in real time. The system 100 includes one or more client computing devices 105 communicatively connected to a central server 120. It is to be contemplated for a person skilled in the art that a system environment can have any client computing devices 105 and may have multiple systems 100 connected to each other through a communication medium 170.

The central server 120 includes a first processor 122, a first memory 123 and one or more predetermined algorithms 124, at least including an ingestion module 124a, having one or more programming instructions 125 embodied thereon, adapted to be implemented by the first processor 122. The central server 120 further includes a reporting unit 126, provided in the form of a display unit, adapted to display an analysis of the ingested data in accordance with the one or more predetermined algorithms 124.

The central server 120 further includes a central repository 127 to store the plurality of historical data-sets 167 and/or the ingested dataset 150, which may also be pushed towards a backend server 140. In some embodiments, the central repository 127 is positioned within the central server 120 itself, as an internal storage. In a preferred embodiment, the central repository 127 is remote to the central server 120 and works in a cloud-based environment. However, in other embodiments, the central repository 127 may be positioned in any possible configuration, as known in the art.

The central server 120 includes a first communication interface 112 for enabling a connection with the one or more client computing devices 105 and a second communication interface 114 adapted to enable communication thereof with the back-end server 140 through the communication medium 130. As used herein, ‘the communication medium 130’ includes a local area network (LAN), a wide area network (WAN), a metropolitan area network (MAN), a virtual private network (VPN), an enterprise private network (EPN), Internet, and a global area network (GAN).

The first communication interface 112 is generally adapted to communicatively connect the client computing devices 105 with the central server 120. In a preferred embodiment, the first interface 112 is a wireless communication interface, including but not limited to LAN, USB, and the like. In some embodiments, the first interface 112 is a wireless communication interface, generally in the form of a Wi-Fi interface, adapted to communicate with the ultrasound devices 105 through the communication medium 130, generally in the form a network selected from one or more of but not limited to a WAN, Internet, Intranet, other Cellular services (2G/4G or NB-IoT), and the like.

The second communication interface 114 is generally adapted to communicatively connect the central server 120 to the back-end server 140 through the communication medium 130. In a preferred embodiment, the second interface 114 is a high energy communication interface, generally in the form of a Wi-Fi interface, adapted to communicate with the back-end server 140 through the communication medium 130, generally in the form a network selected from one or more of but not limited to a WAN, Internet, Intranet, other Cellular services (2G/4G or NB-IoT), and the like.

The back-end server 140 is generally a computing unit having a second processor, a second memory, one or more data-receiving components adapted to receive datasets from a plurality of central servers.

The input healthcare data includes data-sets 145 pertaining to at least data in a predetermined format, received from one or more of plurality of computing devices 105 and may also include other data-sets such as information from any out of the system sources such as claims data files.

The historical data sets may be received from one or more of plurality of data sources 102 (for example, Data Source1 and Data Source2) and unknown data sources (for example, Data Source3, Data Source4-Data Source ‘N’). Initially, the plurality of claims data is received from the plurality of known data sources (Data Source1 and Data Source2) in the form of historical data or past data or known data. For example, historical claims data of the previous 2-3 years may be received for analysis by the central server 120. The historical claims data for any year may contain monthly claims data files. Therefore, from one known data source there may be received, for example, at least 24 claims files for two years of historical data and at least 36 files for a three-year historical data for performing analysis. Further, there may be multiple claims data files in a month. The claims data may contain unique identity information represented by a ‘claimID’ for an encounter between a patient and a healthcare service provider. For each unique ‘claimID’ there may be associated multiple data values inside its rows and columns.

The claims data files typically contain insurance claims data to be audited and processed for settlements. The claims data is arranged in multiple rows and columns in a claims data file. Each column of the claims data files is given a column name by the respective data source 102. The claims data may be recorded by accumulating various information including details of first and second party to the insurance claims. The claims data may be recorded in various formats, wherein different claims data files may have different data structure and different column names. Each column of a claims data file may have different values as described above. The different column names of the claims data files may be chosen because these files come from a plurality of different data sources 102, wherein each data source 102 may use a specific format for recording relevant information in its respective files. Receiving a plurality of claims data files in multiple different formats makes the process of claims mapping challenging and time consuming.

The central server 120 further includes a machine learning based processing sub-module 124b having a plurality of second programming instructions 160. Particularly, the machine learning based processing sub-module 124b is configured to processes the received data sets 145 in accordance with the one or more second programming instructions 160 so to determine a learning model that may upgrade the ingestion model 124a so as to determine, identify, assess, rank, and determine a quantitative or qualitative value or level of diagnosis/decisions events based on known, anticipatory, historical, and/or other data.

The central repository 127 including the plurality of datasets 145, 167 are constantly upgraded on the basis of one or more learning models selected from but not limited to Natural language processing (NLP), Deep Learning, Machine Learning, statistical learning model, and the like.

In an embodiment of the present disclosure, one or more predetermined algorithms 124, includes an ingestion module 124a, the machine-learning module 124b, an analysis module, and a validation module 124c, including the programming instructions 125 based on a deep learning model wherein the model is particularly upgraded on the basis of datasets stored within the central repository 127, including received datasets 145, decision datasets 167 and the like. The ingestion module 124a further includes one or more mapper submodules 134 including a table mapper sub-module 134a adapted to filters one or more tables in decision data sets 167, a column mapper sub-module 134b adapted to identify columns within the filtered data-sets, a row mapper sub-module 134c adapted to identify the row values within the filtered table, and a group schema sub-module 134d adapted to identify group schema rules for the ingestion data-pattern. Further, the validation module 124c includes a validation sub-module 134e adapted to identify and store row level data handling rules for validating quality of data.

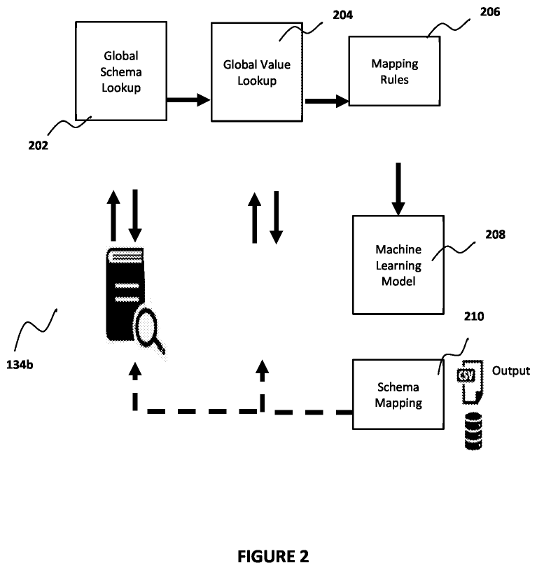

FIG. 2 illustrates the functionality of the mapper sub-module 134 according to an embodiment of the present subject matter. As illustrated, the group schema submodules 134d is utilized to perform the actions of mapper submodules namely, table mapper submodule, column mapper submodule, and the row mapper submodule. Particularly, it is utilized to map the input healthcare data in accordance with the data pattern identified on the basis of the historical claim data tables, columns, and rows respectively. In some embodiments, the group schema submodule includes a Global Value Lookup 204 in addition to mapping code 206. The mapping codes 206 includes various rules for performing the mapping of input healthcare data to the target schema based on the identified data pattern and is adapted to performs the mapping in accordance with the tables, rows and columns by first looking into the tables to filter relevant tables, and then looking into each column of the input healthcare data to identify the data signature for each of the columns followed by mapping data values for each row within the columns. This step also involves analyzing the data values to flag outliers, which in turn helps in identifying and removing non-relevant/invalid mappings. The output received on the basis of mapper sub-module 134 is further adapted to update the Global Schema sub-module 134d and acts as a feedback loop for improving the mapping accuracy of the system over time.

According to an embodiment of the present disclosure, the machine learning model 208 may use an exhaustive list of features computed on the historical claims data. It may analyze the received multiple claims data files over a period of time and based on the analysis and identifies specific data patterns and insight data trends. These features and patterns are then used to train the machine learning models (Supervised Approach, Ensemble) to predict possible matches between incoming columns and the target schema. The system also employs Machine Learning Classification Models (Supervised Learning, Ensemble). A classification uses features (from a list of 22 in-house created features) for predicting the name of the column to be mapped. These features have been created using the historical claims data. Some of these features are statistical while other features are contextual.

Further, the machine learning model and/or the deep learning model includes a learning engine adapted to run a selected model (e.g., deep learning model, Random Forest, multi linear regression, Multilayered, feed-forward neural networks, statistical model, or the like) on the data sets 145, 167, and partitions them into either a training dataset or a testing dataset. In a preferred embodiment, the partitioning may apply an 80/20 split between the training dataset and the testing dataset, respectively.

Thereafter, the learning engine operates to then run the selected model on the training dataset to obtain a resulting output from the model. For example, in a preferred embodiment, the selected model is the Multilayered, feed-forward neural networks, with a Tensor flow backend to build and train the neural networks.

The learning engine then selects and tunes other model arguments of the training dataset to establish an error percentage. Once the error percentage (i.e., accuracy) is established, the learning engine applies a ten-fold cross validation to establish a model stability of the selected model. Further, the learning engine operates dynamically by dynamically selecting the model arguments for each run of the selected model.

Further, the learning engine operates a final model run on the testing dataset to confirm the accuracy and/or fit of the selected model are within client acceptable limits. When the accuracy and/or fit of the selected model is not within the client's acceptable limits or when there are more models left for consideration, a next model may be selected to begin the testing process over again. When the accuracy and/or fit of the selected model is determined to be within the client acceptable limits or when there are no more models left for consideration, the selected model is established for use to ingest data sets 145 and/or identification of ingestion data patterns and/or validation of ingested data 145, received from the one or more client computing devices 105.

The components shown here, their connections and relationships, and their functions, are meant to be exemplary only, and are not meant to limit implementations described and/or claimed in this document.

In a preferred embodiment, the central server 120 is a computing device having a processor, memory, a storage device, a high-speed interface connecting to memory and high-speed expansion ports, and a low-speed interface connecting to low speed bus, one or more input/output (I/O) devices. Each of the components are interconnected using various busses and may be mounted on a common motherboard or in other manners as appropriate.

The processor may communicate with a user through control interface [not shown] and display interface coupled to a display. The display may be, for example, a TFT LCD (Thin-Film-Transistor Liquid Crystal Display) or an OLED (Organic Light Emitting Diode) display, or other appropriate display technology. The display interface may comprise appropriate circuitry for driving the display to present graphical and other information to a user. The control interface may receive commands from a user and convert them for submission to the processor. In addition, an external interface in the form of data-receiving component may be provided in communication with processor, so as to enable near area communication of the central server with other central servers within the system 100. External interfaces may provide, for example, for wired communication in some implementations, or for wireless communication in other implementations, and multiple interfaces may also be used.

The central server is shown as including the memory. The memory may store the executable programming instructions 160. The executable programming instructions 160 may be stored or organized in any manner and at any level of abstraction, such as in connection with one or more applications, processes, routines, procedures, methods, functions, etc.

As used herein, ‘client computing device’ is a smart electronic device capable of communicating with various other electronic devices and applications via one or more communication networks. Examples of said user devices include, but are not limited to, a wireless communication device, a smart phone, a tablet, a desktop, a laptop, etc. The client computing device comprises: an input unit to receive one or more input data; an operating system to enable the user device to operate; a processor to process various data and information; a memory unit to store initial data, intermediary data and final data pertaining to claims data; and an output unit having a graphical user interface (GUI).

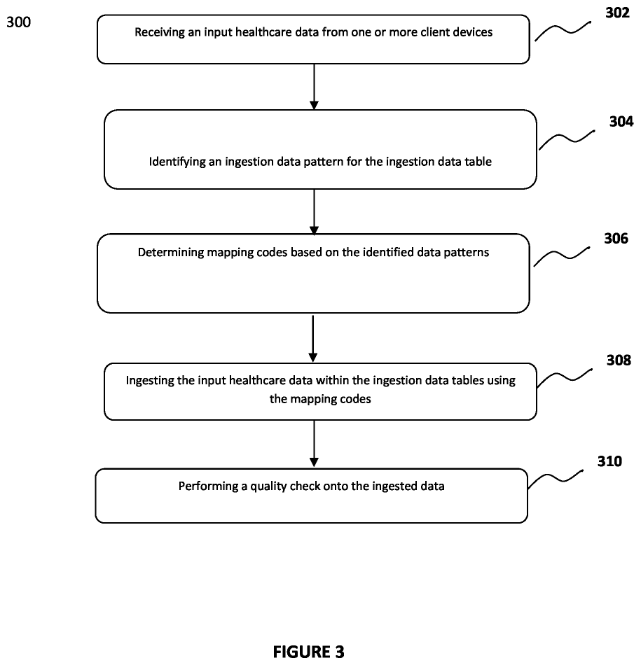

FIG. 3 illustrates a flow chart of a method of automatically ingesting the input healthcare data within the unified database 150 in real time, according to the present disclosure. The method starts at step 302 where an input healthcare data is received at the central server 120 from one or more of the client computing devices 105. The method then proceeds to step 304.

At step 304, the ingestion module 124a identifies an ingestion data pattern for the ingestion data table. The received data sets are ingested within the unified ingestion data table according to the ingestion data pattern. The process of identifying the ingestion data-pattern is described in method 400, utilizing the historical claim data sets/decision data sets 167, received from the plurality of data sources 102.

Once the data-pattern is identified at step 304, the method then proceeds to step 306 where one or more mapping codes are determined based on the identified data pattern. The mapping codes are used to configure and/or upgrade the mapper submodules, namely the table mapper submodule, the column mapper submodule, and the row mapper submodules, wherein one or more rules for tables and schema are predefined. The schema is configured based on one or more predefined rules and/or the plurality of historical claims data. The schema substantially includes various possible measures required for providing quick mapping of claims data being received from different data sources 102 in different formats.

Thus, the mapping codes are determined to automate mapping of the claims data that are being received in different formats by mapping the current incoming claims data files to a target file. The claims data files typically contain insurance claims data to be audited and processed for settlements. The claims data is arranged in multiple rows and columns in a claims data file. Each column of the claims data files is given a column name by the respective data source 102. The claims data may be recorded by accumulating various information including details of first and second party to the insurance claims.

The method then proceeds to step 308 where the input healthcare data is ingested within the ingestion data tables, in accordance with the ingestion data pattern by using the mapping codes determined at step 306. The mapping codes and the schema are used for executing data mapping operations for each of the received one or more columns and associated values, thereby obtaining data mapping results, which are stored in the ingestion data table. Once the mapping is done, the data gaps are checked and finally the data is ingested by writing the SQL ingestion query. According to one embodiment of the disclosure, the mapping of the claims data may be performed by executing two level checks. In the first level, partial similarities between the column names of source and target files are checked. The schema is associated with a mapping list which contains all mapping information and guidelines for applying a particular mapping relationship on the raw data of the claims data files. The mapping list is used to identify which column name of the received claims data from any data source 102 must be mapped to which column of the schema. Thus, by using the mapping list, particular column names from the source are mapped to that of the target files and the first level check is performed. The second level check is performed by reading all the column names, finding an exact match between the source and the target column names, and also accessing the values associated with each column name for the source and target data files.

The method then proceeds to step 310 where the validation sub-module is utilized to perform a quality check onto the ingested data, and monitor if the ingestion is done in accordance with the identified data-pattern. The validation module is configured to monitor each of one or more rows of adjudicating claims data in order to identify whether the associated values are repeating or changing on a periodic basis. Generally, a library of historical claims data and the mappings are stored in a global schema sub-module 134d and a row level data handling rules are stored in a validation sub-module 134e, each being updated after every mapping process and being used in all the subsequent mappings.

The steps of the method 300 are performed in real time, and therefore the method dynamically and continuously keeps on upgrading the data-patterns, and according the mapping codes, thereby keeping the ingestion upgraded in accordance with any update within the data-sets over a period of time. At each step of the method 300, the central server 120 is configured to select at least one of a predetermined algorithm 124 for processing the data-sets 145. Such a selection may be performed automatically. Further, in such embodiments, the central server 120 may select one or more additional algorithms for performing multiple tasks, either in combination or otherwise sequentially one after the other, or otherwise in any other possible order, as may be applicable.

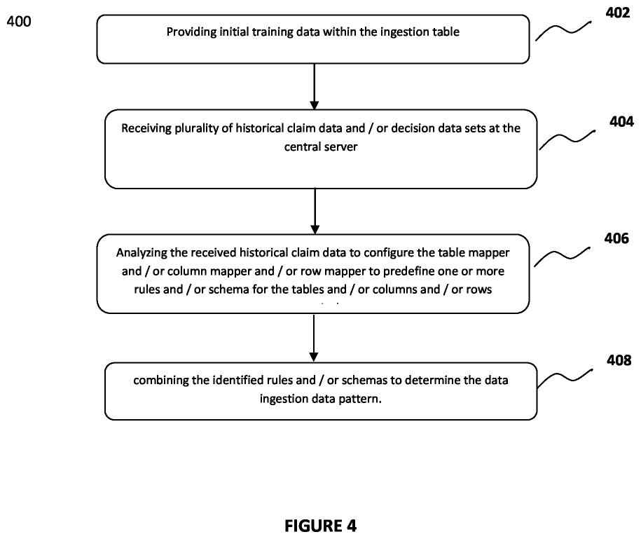

FIG. 4 illustrates a flow chart of a method of identifying ingestion data-pattern to input the healthcare data within the unified database 150 in real time, according to the present disclosure. The method 400 starts at step 402 where an initial training data is provided within the ingestion table. Generally, the machine learning model and/or the deep learning model includes a learning engine adapted to run a selected model on the data set 167 and partitions the data set into either a training dataset or a testing dataset. In a preferred embodiment, the partitioning may apply an 80/20 split between the training dataset and the testing dataset, respectively. The method then proceeds to Step 404 where the plurality of historical claim data and/or decision data sets 167 are received at the central server 120, wherein the decision data sets are required for the identification of data pattern.

At Step 406, the received historical claim data is analyzed to configure the table mapper and/or column mapper and/or row mapper to predefine one or more rules and/or schema for the tables and/or columns and/or rows respectively. The table mapper uses text similarity (lexical, semantic), distancing algorithms, and metadata based analysis based on the table names and the dictionaries created using the historical mappings.

The column mapper uses frequency distribution, word embeddings, outlier detection, knowledge-based similarity, and string-based similarity for the values present inside the input columns. The column mapper also employs clustering algorithms for clustering the similar type attributes.

Further, at Step 408, the identified rules and/or schemas combined to determine the data ingestion data pattern.

According to an embodiment of the present disclosure subject matter, the validation module 124c validates the data mapping results generated by the ingested module 124a. The validation module 124c also monitors each of the one or more rows of adjudicating present claims data to identify how the associated values are repeating or changing on a periodic basis. There may be claims data files that are received every month. The claims data may contain unique identity information represented by a ‘claimID’ for a particular patient or healthcare service provider. Every month, for each unique ‘claimID’, there may be received row values against the several columns. In some scenarios, overwriting of row values is performed by replacing older months' data with latest records. In other cases, however, overwriting of data is not needed and all the records are to be maintained. The validation module 124c checks the row values on a per claimID-basis to identify whether the previous data or values must be retained or overwritten based on the claims data pattern.

The validation module 124c takes the claims data pattern of any unknown data source and further updates the Validation schema module 134c, which acts as a feedback loop for improving the validation accuracy of the system over time. The system is designed in such a way that for every mapping that happens through the system, flows back to the global knowledge-base, which ultimately acts as a feedback loop for improving the accuracy of the system.

According to an embodiment, the system 100 is exemplified with a client architecture system where the central server 120 may be in the form of a mobile application. The mobile application in such instances, includes a front-end user interface that can run off a standard web-browser on desktop environments, or a mobile based smartphone or tablet versions (for Android and iOS); and a backend server 140 which can be a lightweight workstation machine that will collect and process the datasets received from one or more central servers 120.

Advantageously, such an accurate and dynamic ingestion of a plurality if types of data format within the ingestion database, is particularly beneficial in providing real time data ingestion, on the basis of a continuously upgraded data pattern, while avoiding any errors within the unified databases. Further, the system 100 connects the physical and digital worlds by automating, collecting, and storing critical data, creating frictionless workflows to automate healthcare insurance data processing.

Moreover, since the system 100 of the present subject matter is able to communicate via various possible communication interfaces known in the art, it provides flexibility to the organizations/facilities to choose the technology backhaul dependent on existing site infrastructure or requirements. Therefore, an infrastructure upgrade within the facility is not required.

The method and system according to the present disclosure combines a variety of data types from a plurality of data sources within a single unified database which can be accurately utilized by the insurance company to manage the claims, and other insurance related processes for its users.

It is noted that various connections are set forth between elements in the description and in the drawings (the contents of which are included in this disclosure by way of reference). It is noted that these connections in general and, unless specified otherwise, may be direct or indirect and that this specification is not intended to be limiting in this respect. In this respect, a coupling between entities may refer to either a direct or an indirect connection.

Various embodiments of the disclosure have been disclosed. However, it should be apparent to those skilled in the art that modifications in addition to those described, are possible without departing from the inventive concepts herein. The embodiments, therefore, are not restrictive, except in the spirit of the disclosure. Moreover, in interpreting the disclosure, all terms should be understood in the broadest possible manner consistent with the context. In particular, the terms “comprise” and “comprising” should be interpreted as referring to elements, components, or steps, in a non-exclusive manner, indicating that the referenced elements, components, or steps may be present, or utilized, or combined with other elements, components, or steps that are not expressly referenced.

The disclosed methods and systems, as illustrated in the ongoing description or any of its components, may be embodied in the form of a computer system. Typical examples of a computer system include a general-purpose computer, a programmed microprocessor, a micro-controller, a peripheral integrated circuit element, and other devices, or arrangements of devices that are capable of implementing the steps that constitute the method of the disclosure.

The computer system comprises a computer, an input device, a display unit, and the Internet. The computer further comprises a microprocessor. The microprocessor is connected to a communication bus. The computer also includes memory. The memory may be Random Access Memory (RAM) or Read Only Memory (ROM). The computer system further comprises a storage device, which may be a hard-disk drive or a removable storage drive, such as, a floppy-disk drive, optical-disk drive, and the like. The storage device may also be a means for loading computer programs or other instructions into the computer system. The computer system also includes a communication unit. The communication unit allows the computer to connect to other databases and the Internet through an input/output (I/O) interface, allowing the transfer as well as reception of data from other sources. The communication unit may include a modem, an Ethernet-card, or other similar devices, which enable the computer system to connect to databases and networks, such as, LAN, MAN, WAN, and the Internet. The computer system facilitates input from a user through input devices accessible to the system through an I/O interface.

In order to process input data, the computer system executes a set of instructions that are stored in one or more storage elements. The storage elements may also hold data or other information, as desired. The storage element may be in the form of an information source, or a physical memory element present in the processing machine.

The programmable or computer-readable instructions may include various commands that instruct the processing machine to perform specific tasks, such as steps that constitute the method of the disclosure. The systems and methods described can also be implemented using only software programming or using only hardware or by a varying combination of the two techniques. The disclosure is independent of the programming language and the operating system used in the computers. The instructions for the disclosure can be written in all programming languages including, but not limited to, “C”, “C#”, “C+”, “C++”, “Embedded C”, “Visual C++,” Java “, “Python” and “Visual Basic”. Further, the software may be in the form of a collection of separate programs, a program module containing a larger program or a portion of a program module, as discussed in the ongoing description. The software may also include modular programming in the form of object-oriented programming. The processing of input data by the processing machine may be in response to user commands, the results of previous processing, or from a request made by another processing machine. The disclosure can also be implemented in various operating systems and platforms including, but not limited to, “iOS”, “Mac” “Unix,” “DOS,” “Android,” “Symbian,” and “Linux.”

The programmable instructions can be stored and transmitted on a computer-readable medium. The disclosure can also be embodied in a computer program product comprising a computer-readable medium, or with any product capable of implementing the above methods and systems, or the numerous possible variations thereof.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor and can be implemented in a high-level procedural and/or object-oriented programming language, and/or in assembly/machine language. As used herein, the terms “machine-readable medium” and “computer-readable medium” refer to any computer program product, apparatus and/or device (e.g., magnetic discs, optical disks, memory, Programmable Logic Devices (PLDs)) used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

A person having ordinary skills in the art will appreciate that the system, modules, and sub-modules have been illustrated and explained to serve as examples and should not be considered limiting in any manner. It will be further appreciated that the variants of the above disclosed system elements, or modules and other features and functions, or alternatives thereof, may be combined to create other different systems or applications.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (“LAN”), a wide area network (“WAN”), and the Internet.

The claims can encompass embodiments for hardware, software, or a combination thereof.

Although various implementations have been described in detail, other modifications are possible. Moreover, other mechanisms for performing the systems and methods described in this document may be used. In addition, the logic flows depicted in the figures may not require the particular order shown, or sequential order, to achieve desirable results. Other steps may be provided, or steps may be eliminated, from the described flows, and other components may be added to, or removed from, the described systems. Accordingly, other implementations are within the scope of the following claims.

While the preferred embodiments of the present disclosure have been described hereinabove, it should be understood that various changes, adaptations, and modifications may be made therein without departing from the spirit of the disclosure and the scope of the appended claims. It will be obvious to a person skilled in the art that the present disclosure may be embodied in other specific forms without departing from its spirit or essential characteristics. The described embodiments are to be considered in all respects only as illustrative and not restrictive.

We claim:

1. A method for automatically and dynamically ingesting healthcare data in a real-time, the method comprising:
receiving, at a central server, an input healthcare data from one or more client computers connected thereto;
identifying an ingestion data pattern for an ingestion table adapted to ingest the input healthcare data on a basis of a plurality of historical claims data;
generating one or more mapping codes on a basis of identified data-patterns, the mapping codes comprising a table mapper sub-module, a column mapper sub-module, and a row mapper sub-module; and
ingesting the input healthcare data in accordance with the identified data pattern by using the generated one or more mapping codes, wherein ingesting the input healthcare data is performed in a real-time, whereby the ingestion data pattern is dynamically updated, and the ingested data is monitored to validate a mapping of the input healthcare data within the ingestion table.

2. The method of claim 1, wherein the input healthcare data comprises insurance claim data.

3. The method of claim 1, wherein identifying the ingestion data pattern comprises:
providing an initial training data within the ingestion table;
receiving, at the central server, at least one historical claim data from one or more of a plurality of data sources connected thereto;
analyzing the received at least one historical claim data to configure at least one of the table mapper sub-module, the column mapper sub-module, or the row mapper sub-module to predefine at least one of rules or schema for the ingestion table, or columns or rows of the ingestion table, respectively; and
combining the at least one of rules or schemas to determine the data ingestion data pattern.

4. The method of claim 3, wherein configuring at least one of the table mapper sub-module, the column mapper sub-module, or the row mapper sub-module comprises performing a two-level check including:
performing a first level check to identify similarities in table names; and
performing a second level check to identify similarities in at least one of a column name or a row name along with associated values with respect to the at least one of the rules or schema.

5. The method of claim 3, wherein each of the initial training data and the plurality of historical claims data stored within the plurality of data sources comprises historical insurance claim data stored in multiple data formats.

6. The method of claim 1, further comprising upgrading an ingestion module used for identifying the ingestion data pattern for the ingestion table with a machine learning based module at a cloud server, the cloud server communicatively connected to a plurality of ingestion modules of a plurality of central servers.

7. A system for automatically and dynamically ingesting healthcare data in a real-time, the system comprising:
a central server communicatively coupled to at least one client computer via a first communication network, and a plurality of data sources via a second communication network, the server comprising:
a receiving unit adapted to receive an input healthcare data from the at least one client computer;
a memory comprising one or more ingestion databases adapted to store a plurality of healthcare data in a predetermined data pattern; and
one or more processors for processing the input healthcare data in accordance with an ingestion module, the ingestion module being stored in the memory and, being executable by the one or more processors to ingest the input healthcare data into the one or more ingestion databases, the ingestion module comprising instructions for:
accessing a plurality of historical claims data from the one or more of plurality of data sources;
identifying the predetermined data pattern for an ingestion table on a basis of the plurality of historical claims data;
generating one or more mapping codes on a basis of identified predetermined data patterns, the one or more mapping codes comprising a table mapper sub-module and a column mapper sub-module; and
ingesting the healthcare input data in accordance with the identified predetermined data pattern using the one or more mapping codes; and
wherein the input healthcare data is processed in a real-time whereby data pattern is dynamically updated, and the ingested input healthcare data is monitored to validate a mapping of the input healthcare data within the ingestion database.

8. The system of claim 7, wherein the healthcare data is insurance claim data.

9. The system of claim 7, wherein the plurality of historical claims data from the plurality of data sources comprises historical insurance claim data stored in multiple data formats.

10. The system of claim 7, wherein validation of the mapping is performed by a validation sub-module stored within the memory of the central server.

11. The system of claim 7, wherein the predetermined data pattern further comprises an ingestion data pattern, wherein the ingestion module further comprises instructions for identifying the ingestion data pattern, the instructions comprising:
providing an initial training data within the ingestion table of the ingestion database;
receiving, at the central server, at least one historical claim data from the plurality of historical claims data from one or more of a plurality of data sources;
analyzing the received at least one historical claim data to configure at least one of the table mapper sub-module, the column mapper sub-module, or a row mapper sub-module to predefine at least one of rules or schema for the ingestion table, or columns or rows of the ingestion table, respectively; and
combining the at least one of rules or schemas to determine the data ingestion data pattern.

12. The system of claim 11, wherein instructions for configuring at least one of the table mapper sub-module, the column mapper sub-module, or the row mapper sub-module comprises instructions performing a two-level check, the instructions including:
performing a first level check to identify similarities in table names; and
performing a second level check to identify similarities in at least one of a column name or a row name along with associated values with respect to the at least one of the rules or schema.

13. The system of claim 11, wherein each of the initial training data and the plurality of historical claims data stored within the plurality of data sources comprises historical insurance claim data stored in multiple data formats.
